(19) United States
(12) Patent Application Publication
(10) Pub. No.: US 2012/0121700 A1
(43) Pub. Date: May 17, 2012

(54) PHARMACEUTICAL FORMULATIONS COMPRISING VALGANCICLOVIR

(76) Inventors: Vaibhav Dubey, Sagar (IN); Mamta Mishra, Hyderabad (IN); Ramakant Gundu, Aurangabad (IN); Navin Vaya, Hyderabad (IN); Harshal Prabhakar Bhagwatwar, Hyderabad (IN)

(21) Appl. No.: 13/295,294

(22) Filed: Nov. 14, 2011

Related U.S. Application Data
(60) Provisional application No. 61/430,591, filed on Jan. 7, 2011.

(30) Foreign Application Priority Data
Nov. 15, 2010 (IN) 3429/CHE/2010

(51) Int. Cl. A61K 9/48; A61K 9/28; A61P 31/22; A61K 31/522

(52) U.S. Cl. 424/452; 514/263.38; 424/465

(57) ABSTRACT

Pharmaceutical formulations prepared by granulating valganciclovir or a salt thereof, using a nonaqueous solvent or hydro-alcohol.

### INTRODUCTION

In aspects, the present disclosure relates to pharmaceutical formulations comprising valganciclovir, processes for preparing pharmaceutical formulations, and methods of using the formulations. In embodiments, the present disclosure relates to solid dosage forms comprising valganciclovir hydrochloride that are manufactured using a solvent granulation technique.

Valganciclovir hydrochloride, a prodrug of ganciclovir, is used in the treatment of cytomegalovirus (CMV) retinitis in patients with acquired immunodeficiency syndrome (AIDS), and for the prevention of CMV disease in kidney, heart, and kidney-pancreas transplant patients who are at high risk.

Valganciclovir hydrochloride is a hydrochloride salt of the L-valyl ester of the drug ganciclovir. It has a chemical name L-valine, 2-[(2-amino-1,6-dihydro-6-oxo-9H-purin-9-yl)methoxy]-3-hydroxypropyl ester, monohydrochloride, and is the active ingredient in a tablet product and a powder for oral solution, sold as VALCYTE®. The tablet product contains 450 mg of valganciclovir (as 496.3 mg of valganciclovir hydrochloride) and has the inactive ingredients microcrystalline cellulose, povidone K-30, crospovidone, stearic acid, and a pink film coating.

The structural formula for valganciclovir hydrochloride is as follows:

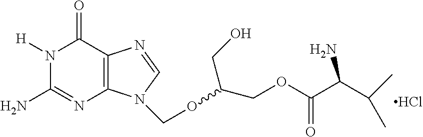

U.S. Pat. No. 6,083,953 discloses valganciclovir hydrochloride and processes for its preparation. The patent teaches that valganciclovir hydrochloride was developed with a view to improve the bioavailability of gancilcovir, which has a poor bioavailability when administered orally. High oral doses of ganciclovir are required to achieve a therapeutic concentration in the blood. Valganciclovir hydrochloride, when administered orally, is reported to have better bioavailability than ganciclovir given orally.

U.S. Patent Application Publication No. 2007/0292499 discloses the preparation of solid dosage forms containing amorphous valganciclovir, using a dry process with a view to improve the solubility and bioavailability of valganciclovir.

The amorphous form of a drug sometimes has certain advantages over the crystalline form, for example, amorphous forms can be more soluble or can have a higher rate of solubility in water than crystalline forms and consequently the drug may show improved bioavailability, for example, due to the faster dissolution of the drug in the gastrointestinal fluid. However, in some cases, amorphous forms are more prone to degradation and pose stability problems in a formulation. It is necessary to have polymorphic stability of the active agent during manufacturing and throughout the normal shelf life of the product.

It has been found that amorphous valganciclovir hydrochloride is a very fine and fluffy material, with relatively low bulk and tapped densities. These properties can make it difficult to formulate the material into dosage forms with uniformity of weight, hardness, and other desirable tablet properties. Aqueous granulation using water alone needs to be avoided, as addition of water and its subsequent removal by drying the granules at elevated temperatures may convert the amorphous form to a crystalline form and even present stability issues of the drug due to the presence of residual moisture.

A dry process may not be desirable for a drug with a bulk density less than about 0.2 g/mL, due to poor flow properties of the material leading to non-uniform die filling and subsequent weight variation after direct compression, Further, a dry granulation method often produces a high percentage of fine granules, which can pose problems with the hardness of the tablets or create yield problems.

### SUMMARY

Aspects of the present disclosure relate to solid dosage form compositions comprising valganciclovir, or any pharmaceutically acceptable salts, esters, amides, prodrugs, metabolites, analogs, solvates, hydrates, enantiomers, and derivatives thereof. A reference herein to “valganciclovir” includes all of these. The salt valganciclovir hydrochloride is discussed herein as a representative of valganciclovir.

In an aspect, the present disclosure provides pharmaceutical compositions comprising valganciclovir as an active agent, wherein the compositions are substantially chemically stable during the formulation process and throughout the normal shelf life of the pharmaceutical compositions.

In an aspect, the present disclosure provides pharmaceutical compositions comprising valganciclovir as an active agent, wherein the polymorphic form of the said active agent that is used to make the compositions is substantially retained during the formulation process and throughout the shelf life of the pharmaceutical compositions.

In an aspect, the present disclosure provides pharmaceutical compositions comprising valganciclovir in an amorphous or crystalline form, or mixtures thereof, as an active agent, wherein an amorphous or crystalline form is substantially retained during the formulation process and throughout the shelf life of the pharmaceutical compositions.

In an aspect, the present disclosure provides pharmaceutical compositions comprising valganciclovir in a crystalline form as an active agent, wherein the crystalline form is substantially retained during the formulation process and throughout the shelf life of the pharmaceutical compositions.

In an aspect, the present disclosure provides pharmaceutical compositions comprising valganciclovir in a substantially amorphous or crystalline form, or mixtures thereof, as an active agent, wherein the substantially amorphous or crystalline form or mixtures thereof is substantially retained during the formulation process and throughout the shelf life of the pharmaceutical compositions.

Aspects of the present disclosure relate to processes for preparing pharmaceutical compositions comprising valganciclovir.

In an aspect, the present disclosure provides processes of preparing pharmaceutical compositions comprising valganciclovir or its hydrochloride salt, using organic solvent granulation techniques.

An aspect of the present disclosure relates to the preparation of solid dosage forms comprising valganciclovir or its hydrochloride salt, using non-aqueous wet granulation techniques.

In aspects, the present disclosure relates to non-aqueous wet granulation processes for the preparation of solid dosage forms comprising valganciclovir or its hydrochloride salt and one or more pharmaceutically acceptable excipients.

An aspect of the present disclosure provides processes for the preparation of solid dosage forms comprising valganciclovir or its hydrochloride salt, wherein the processes involve preparing solutions or dispersions of valganciclovir or its hydrochloride salt that are sprayed onto one or more pharmaceutically acceptable excipients, and forming the mixtures into solid dosage forms.

An aspect of the disclosure provides processes for the preparation of solid dosage forms of valganciclovir or its hydrochloride salt, wherein the processes comprise mixing valganciclovir or its hydrochloride salt with one or more pharmaceutically acceptable excipients, granulating the mixture with a non-aqueous or hydro-alcoholic solvent, drying the granules and compressing the dried granules thus obtained into tablets.

In an aspect, processes are provided for the preparation of solid dosage forms of valganciclovir or its hydrochloride salt, wherein the processes comprise dissolving valganciclovir or its hydrochloride salt in a non-aqueous solvent, optionally together with one or more pharmaceutically acceptable excipients, spraying the solution or dispersion onto a pharmacologically inert material to form granules, drying the granules; optionally blending the granules with a lubricant, and compressing the granules into tablets or filling the granules into capsules.

In an aspect, the present disclosure relates to methods of using compositions of the present disclosure comprising valganciclovir or its hydrochloride salt, by administering an effective amount of the composition to a subject in need thereof.

In an aspect, the compositions of the present disclosure are useful in the treatment of cytomegalovirus (CMV) retinitis, or in the prevention of CMV disease.

### BRIEF DESCRIPTION OF THE DRAWINGS

FIG. 1 shows a powder X-ray diffraction (PXRD) pattern of amorphous valganciclovir hydrochloride used in Examples 1-10.

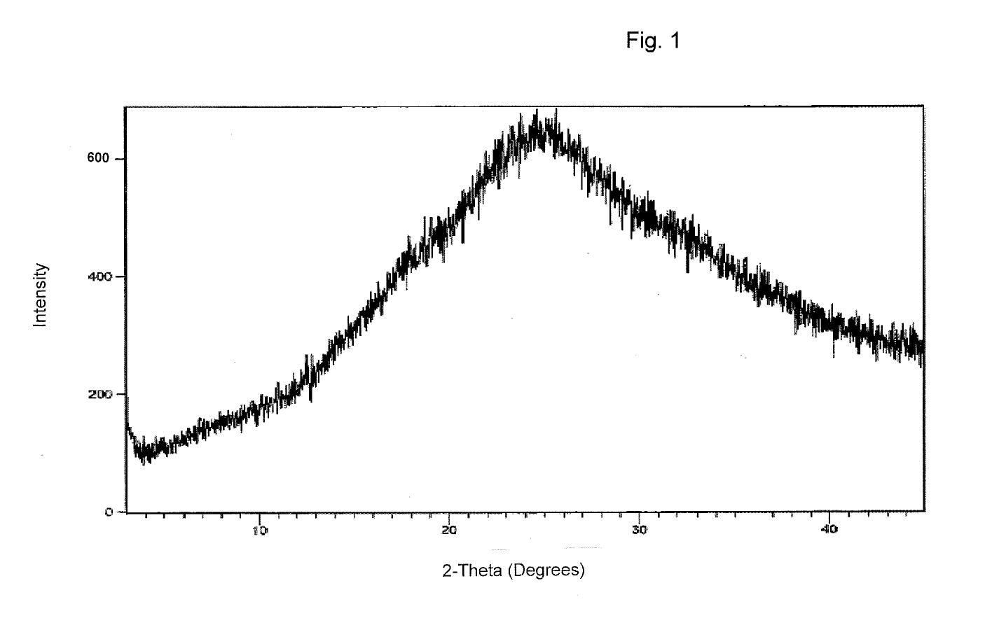

FIG. 2 shows a PXRD pattern of a tablet as prepared in Example 2 (upper curve), and a PXRD pattern of a placebo tablet prepared similarly, but without any drug (lower curve).

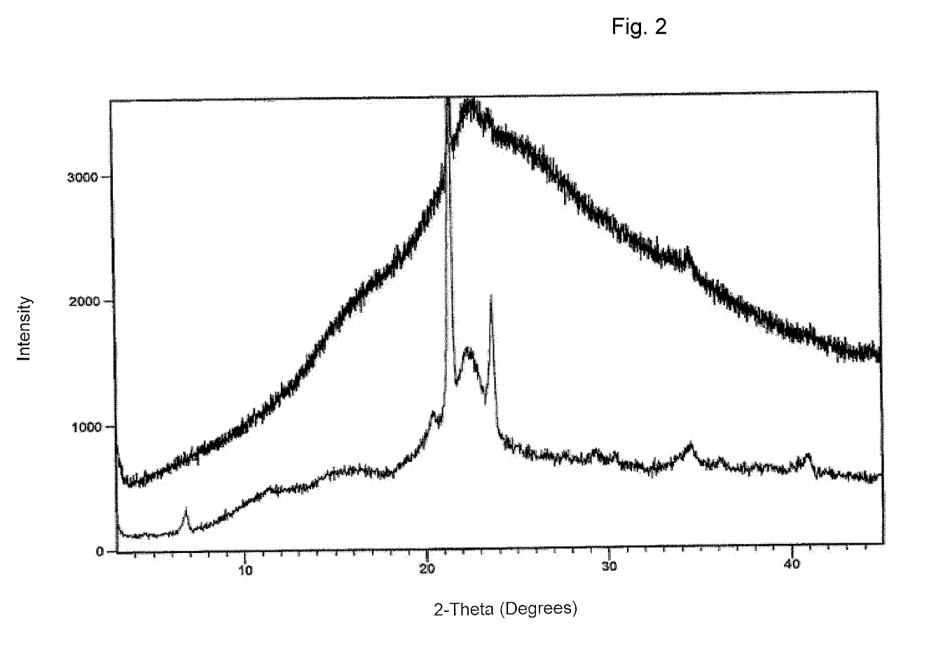

FIG. 3 shows a PXRD pattern of a tablet as prepared in Example 3 (upper curve), and a PXRD pattern of a placebo tablet prepared similarly, but without any drug (lower curve).

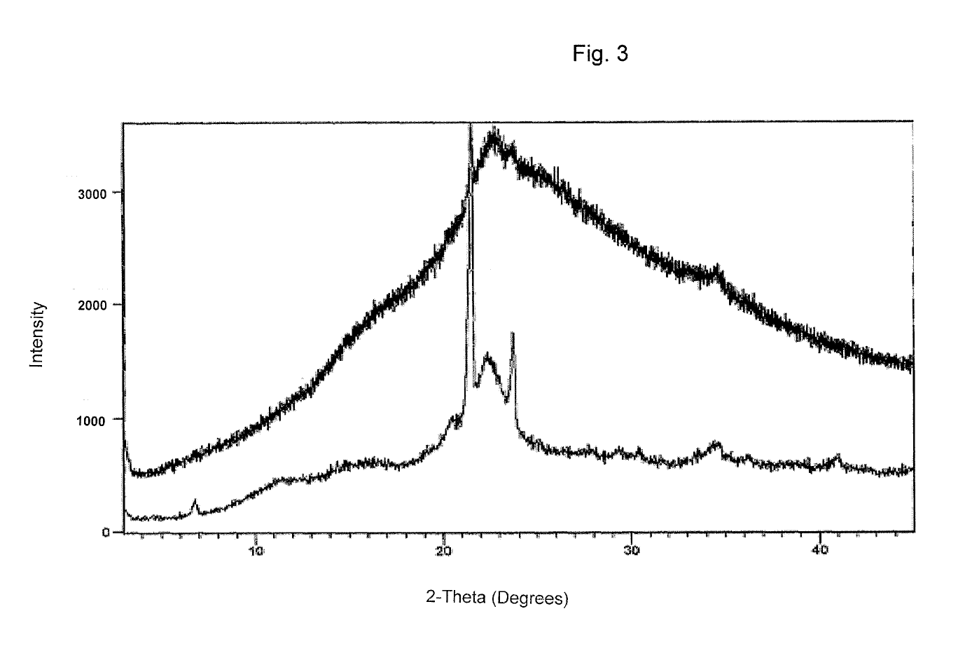

FIG. 4 shows a PXRD pattern of a tablet as prepared in Example 1 (upper curve), and a PXRD pattern of a placebo tablet prepared similarly, but without any drug (lower curve).

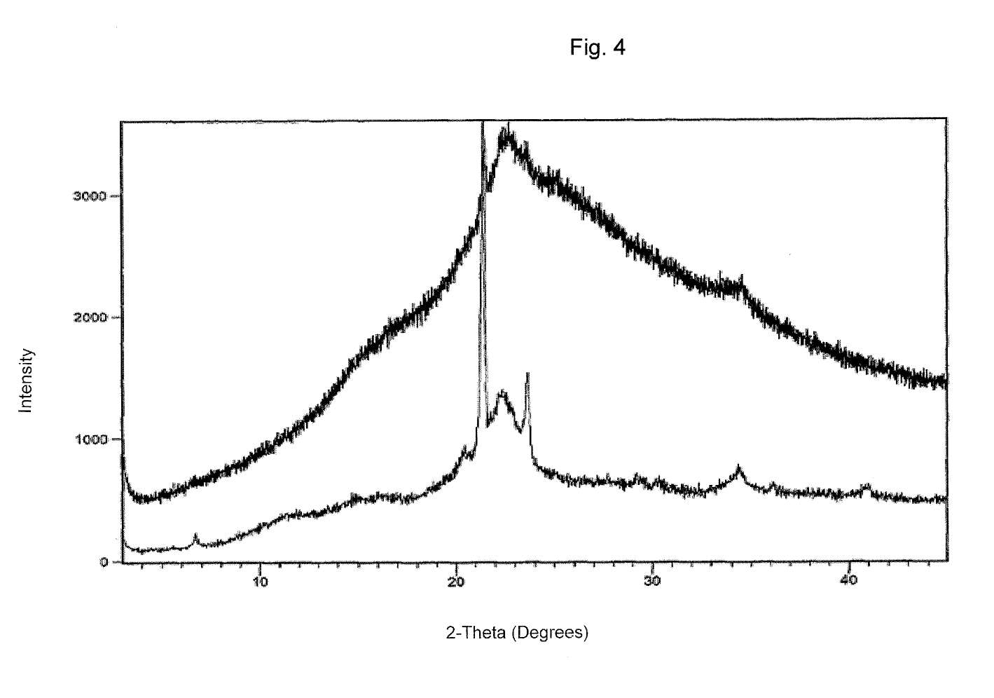

FIG. 5 shows a PXRD pattern of a tablet as prepared in Example 4 (upper curve), and a PXRD pattern of a placebo tablet prepared similarly, but without any drug (lower curve).

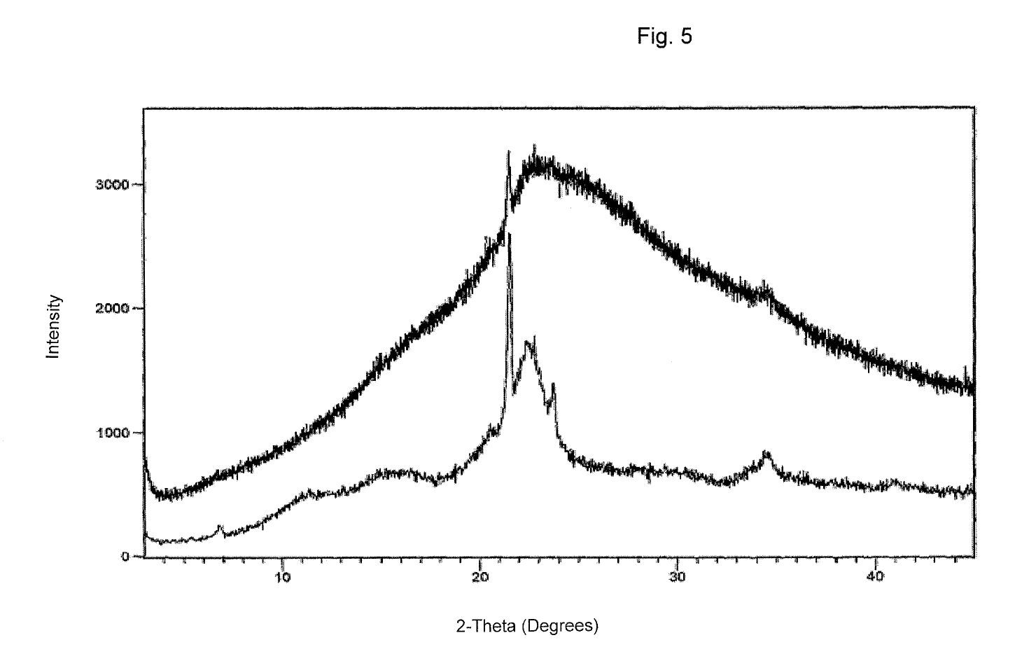

FIG. 6 shows a PXRD pattern of a tablet as prepared in Example 5 (upper curve), and a PXRD pattern of a placebo tablet prepared similarly, but without any drug (lower curve).

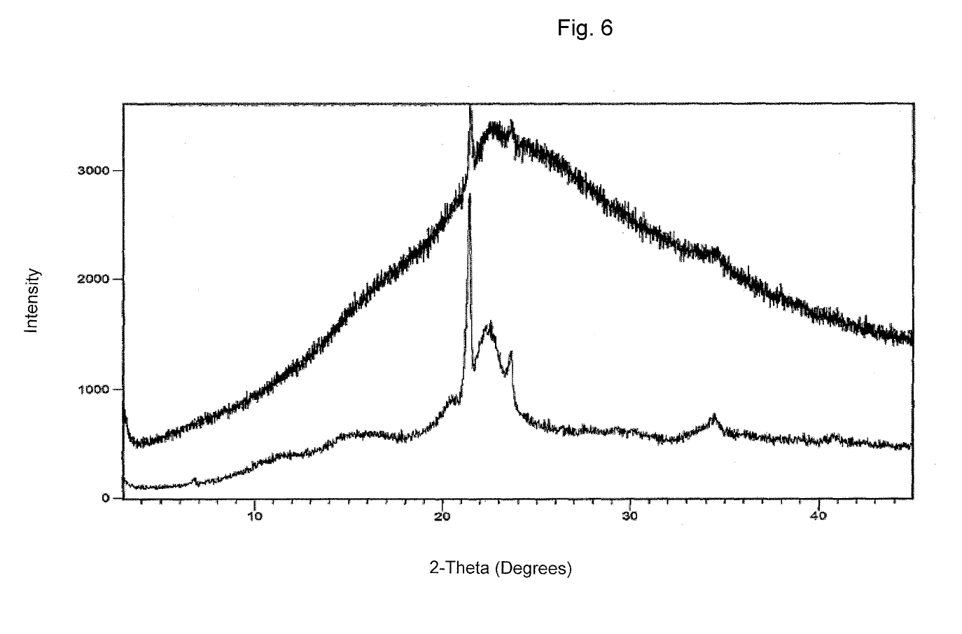

### DETAILED DESCRIPTION

In aspects, the present disclosure provides pharmaceutical formulations comprising the compound valganciclovir, or pharmaceutically acceptable salts, esters, amides, prodrugs, metabolites, analogs, solvates, hydrates, enantiomers, derivatives thereof, processes for preparing compositions, and methods of using the compositions. All of these are referred to herein as “valganciclovir.” For purposes of brevity, the salt valganciclovir hydrochloride is used herein to exemplify various aspects and embodiments, but the scope of the disclosure is not to be limited thereto. When the terms “active agent” and “drug” are used in the present disclosure, it is to be understood that this includes the compound valganciclovir, as well as any of its pharmaceutically acceptable salts, esters, amides, prodrugs, metabolites, analogs, solvates, hydrates, enantiomers, derivatives, and the like.

In certain embodiments, the present disclosure provides solid dosage forms of valganciclovir hydrochloride, manufactured using a non-aqueous solvent or hydro-alcohol granulation technique.

Where a range of values is provided, it is understood that each intervening value, unless the context clearly dictates otherwise, and the upper and lower limits of that range, and any other stated or intervening value in that stated range, are encompassed.

Unless defined otherwise, all technical and scientific terms used herein have the same meaning as commonly understood by one of ordinary skill in the art to which this application belongs. Although any methods and materials similar or equivalent to those described herein can also be used in the practice or testing of the present application, only certain methods and materials are now described.

The percentages of particles with different dimensions that exist in a powder define the particle size distribution. It is represented in certain ways. Particle size is the maximum dimension of a particle, frequently expressed in units of μm. Particle size distributions can be expressed in terms of, D₁₀, D₅₀, and D₉₀, representing the 10th percentile, median or 50th percentile, and the 90th percentile of the particle size distribution, respectively, as measured by volume. Values associated with D₁₀, D₅₀, and D₉₀ are the maximum particle sizes for those fractions. D₅₀ is also known as the median size of particles. It is one of the important parameters representing characteristics of particles in a powder. For a sample, if D₅₀=5 μm, it means that 50% by volume of the particles are smaller than 5 μm. Similarly, if D₁₀=5 μm, 10% by volume of the particles are less than or equal to 5 μm, and if D₉₀=5 μm, 90% by volume of the particles are less than or equal to 5 μm.

The particle sizes of the valganciclovir for the present disclosure can be obtained by any suitable process of synthesis and/or particle size classifying or reducing known in the art, such as using sieving and equipment such as ball mills, jet mills, pulverizers, and fluid energy mills.

In certain embodiments, the disclosure includes pharmaceutical formulations of valganciclovir, wherein the particle size distributions of the valganciclovir active ingredient used are such that D₁₀ is no greater than about 70 μm, D₅₀ is no greater than about 130 μm, and D₉₀ is no greater than about 250 μm.

As used herein, the singular forms “a,” “an,” and “the” include plural referents unless the context clearly dictates otherwise. Thus, for example, reference to “a compound” includes a plurality of such compounds and reference to “a step” includes reference to one or more steps and equivalents thereof known to those skilled in the art, etc.

In embodiments, the present disclosure provides solid dosage forms comprising valganciclovir hydrochloride in amorphous form, prepared using a non-aqueous wet granulation technique. The processes are simple and robust, and do not require high temperature to dry the granules, as is required generally with aqueous wet granulation processes. The amorphous form resists substantial conversion to a crystalline form, thus providing compositions that are polymorphically stable. Such compositions of the present disclosure are advantageous because the physico-chemical properties of the composition, such as dissolution behavior, remain substantially unaltered during a commercially relevant storage period and shelf life. In general, stability is required during manufacturing and for a period of about 6, 12, 18, or 24 months thereafter, in the original packaging at typical ambient storage temperatures. Such stability can be predicted for packaged products, from the results of customary stability testing conditions, such as from storage at 25° C. and 60% relative humidity (RH), “accelerated” conditions such as 40° C. and 75% RH, etc.

Polymorphic forms can be determined using techniques such as PXRD. All X-ray analyses reported herein are obtained using equipment with a copper Kα radiation source.

The term “solid dosage form” as used herein includes formulations such as granules, tablets, coated tablets, and capsules filled with granules or tablets.

The valganciclovir active ingredient in a solid dosage form is present in a prophylactically or therapeutically effective amount. Typically, the drug may comprise from about 1 mg to about 1000 mg, or from about 50 mg to about 800 mg, per unit.

Additionally, other drugs in a prophylactically or therapeutically effective amount can be included in the present dosage form compositions.

In an aspect, the present disclosure relates to non-aqueous wet granulation processes for preparing solid dosage forms comprising valganciclovir or its hydrochloride salt and one or more pharmaceutically acceptable excipients.

Useful pharmaceutically acceptable excipients are those known to persons skilled in the art and may include, but are not limited to, any one or more of fillers, binders, disintegrants, glidants, lubricants, coloring agents, and coating agents. These excipients may be present intragranularly, extragranularly, or both.

Examples of fillers or diluents include microcrystalline cellulose, mannitol, sucrose, lactose, dextrose, calcium carbonate, sorbitol, and the like. A filler may be present in amounts from about 15% to about 90%, or about 20% to 50%, by weight of the dosage form.

Binders include polyvinylpyrrolidones, hydroxypropylcelluloses, hydroxypropyl methylcelluloses, starches and starch based binders, gelatin, gums, and the like. A binder may be present in amounts about 0.1% to about 15%, or about 1% to about 7.5%, by weight of the dosage form.

Disintegrants include crospovidones, croscarmellose sodium, starches, hydroxypropyl celluloses, hydroxypropyl methylcelluloses, gums, sodium starch glycolate, and the like. A disintegrant may be present in amounts about 1% to about 40%, or about 2% to about 20%, by weight of the dosage form.

Lubricants and glidants include talc, colloidal silicon dioxide, magnesium stearate, stearic acid, and sodium stearyl fumarate. Such ingredients may be present in amounts about 0.1% to about 2%, by weight of the dosage form.

Non-aqueous solvents for use in granulation process steps include, but are not limited to, alcohols such as methanol, ethanol, and isopropyl alcohol, halogenated hydrocarbons such as dichloromethane, esters such as ethyl acetate, and any combinations of two or more thereof.

Hydro-alcohols are mixtures of one or more alcohols and water, in volume ratios about 1:50 to about 50:1.

The foregoing lists are not intended to be exhaustive, as those skilled in the art are aware of many other substances that can be used. Also, as is known in the art, some excipient substances can have more than one function in pharmaceutical formulations.

In an aspect, the present disclosure provides processes for preparing pharmaceutical compositions comprising valganciclovir.

In an aspect, the present disclosure provides processes for preparing pharmaceutical compositions comprising valganciclovir, using solvent granulation techniques.

The processes are capable of producing dosage forms with uniformity of weight, as well as sufficient hardness and friability.

In embodiments of processes, valganciclovir hydrochloride is mixed with one or more pharmaceutical excipients such as filler, binder, disintegrant, and lubricant in a suitable blender. The blend is then granulated by spraying or adding a non-aqueous solvent or hydro-alcohol, optionally in combination with a binder or any other excipient. A non-aqueous solvent is primarily an organic solvent, whereas a hydro-alcohol comprises a mixture of an alcohol (e.g., methanol, ethanol, or isopropyl alcohol) and water. The granules are dried and optionally lubricated, then are compressed into tablets, filled into capsules, or packed into sachets. The granules can also be mixed with one or more pharmaceutically acceptable excipients, such as a disintegrant, glidant, etc., before blending with a lubricant.

In embodiments of processes, valganciclovir hydrochloride is dissolved in a solvent such as, but not limited to, an alcohol such as methanol, ethanol, or isopropyl alcohol, a halogenated hydrocarbon such as dichloromethane, or any mixture thereof, and then sprayed onto one or more of pharmaceutical excipients such as a filler, binder, and/or disintegrant to form granules, which are dried, and the dried granules are milled to obtain desired particle sizes. Optionally, extragranular excipients are added and the blend is compressed into tablets.

In embodiments, tablets of the present disclosure are optionally coated using a suitable coating agent or film-forming agent, such as a cellulosic polymer or polyvinyl alcohol, the coating mixtures optionally also containing additives such as a plasticizer, lubricant, etc.

In embodiments, the present disclosure provides pharmaceutical formulations comprising valganciclovir as an active agent, wherein the formulations are substantially chemically stable during the formulation process and throughout the commercially relevant storage life of the pharmaceutical formulations. “Substantially chemically stable” means that a total of valganciclovir-related impurities amounts to less than about 3% of the label valganciclovir content, after storage in closed packages at 40° C. and 75% RH for 3 months.

In embodiments, the present disclosure provides pharmaceutical formulations comprising valganciclovir as an active agent, wherein the polymorphic form of the active agent that is used to make the formulations is substantially retained during the formulation process and throughout the commercially relevant storage life of the pharmaceutical formulations. In embodiments, the formulations can contain other polymorphic forms of valganciclovir, amounting to less than about 30% of the label valganciclovir content, after storage in closed packages at 40° C. and 75% RH for 3 months.

In embodiments, the present disclosure provides pharmaceutical formulations comprising valganciclovir as an active agent in an amorphous form, wherein the amorphous form is substantially retained during the formulation process and throughout the commercially relevant storage life of the pharmaceutical formulations. In embodiments, the formulations can contain crystalline valganciclovir in amounts less than about 30%, or less than about 10%, of the label valganciclovir content, after storage in closed packages at 40° C. and 75% RH for 3 months.

In embodiments, the present disclosure provides pharmaceutical formulations comprising valganciclovir as an active agent in a crystalline form, wherein the crystalline form is substantially retained during the formulation process and throughout the commercially relevant storage life of the pharmaceutical formulations. In embodiments, the formulations can contain other polymorphic forms of valganciclovir, amounting to less than about 30%, or less than about 10%, of the label valganciclovir content, after storage in closed packages at 40° C. and 75% RH for 3 months.

In embodiments, the present disclosure provides pharmaceutical formulations comprising valganciclovir as active agent in a substantially amorphous or crystalline form or mixtures thereof, wherein the original amorphous or crystalline form or mixtures thereof is substantially retained during the formulation process and throughout the commercially relevant storage life of the pharmaceutical formulations. In embodiments, the formulations can contain other polymorphic forms of valganciclovir, amounting to less than about 30%, or less than about 10%, of the label valganciclovir content, after storage in closed packages at 40° C. and 75% RH for 3 months.

A measure of durability of tablets is the test for friability, such as described in Test 1216 “Tablet Friability,” United States Pharmacopeia 29, United States Pharmacopeial Convention, Inc., Rockville, Md., 2005. A friability value of about 1% is usually considered acceptable. Tablets prepared according to embodiments of processes of the present disclosure can have friability less than about 0.8%, or less than about 0.5%.

In embodiments, processes of providing stable solid dosage forms comprising amorphous valganciclovir or its hydrochloride salt involve dissolving the drug in a suitable non-aqueous solvent, spraying onto a pharmacologically inert material, mixing the material thus obtained with one or more pharmaceutically acceptable excipients, drying the granules, and compressing the granules thus obtained into tablets.

In embodiments, processes of this disclosure comprise mixing amorphous valganciclovir or its hydrochloride salt with a filler, binder, and disintegrant, granulating the mixture using a granulating fluid comprising a non-aqueous organic solvent, drying the granules, optionally mixing granules with one or more pharmaceutically acceptable excipients, optionally, mixing with a lubricant, and compressing the mixture to form tablets or filling the mixture into capsules.

In embodiments, processes of this disclosure comprise mixing amorphous valganciclovir or its hydrochloride salt with a filler, binder and disintegrant; granulating the mixture using a granulating fluid comprising a non-aqueous organic solvent; drying the granules; optionally mixing granules with one or more pharmaceutically acceptable excipients; optionally mixing with a lubricant, and compressing the mixture to form tablets. The compressed tablets are coated with a seal coating and then coated with a moisture resistant coating such as an Opadry® product and/or hydrophobic substances such as polymers or waxes or mixtures thereof.

In embodiments of this disclosure are provided compressed tablets of valganciclovir that are coated with a water soluble polymer to form a seal coating, and finally further coated with a moisture resistant coating.

Embodiments of this disclosure provide compressed tablets of valganciclovir that are coated with a polyvinyl alcohol film, upon which a wax or other hydrophobic coating is applied in order to further prevent the degradation of valganciclovir due to penetration of moisture.

Embodiments of this disclosure provide compressed tablets of valganciclovir that are prepared using excipients having a low moisture content, such as below about 10%, 5%, 2%, or 1%, by weight.

In embodiments, formulations of the present disclosure are packaged into suitable containers such as a blister package or a closed bottle, optionally also containing a desiccant and/or an oxygen absorbent.

In aspects, the present disclosure relates to methods of using formulations of the present disclosure comprising valganciclovir, by administering an effective amount of a composition to a subject in need thereof.

In an aspect, compositions of the present disclosure are useful in the treatment of cytomegalovirus (CMV) retinitis, or in the prevention of CMV disease.

Certain specific aspects and embodiments of the disclosure will be further illustrated by the following examples, but these are not to be construed as limiting the scope of the disclosure in any manner.

### EXAMPLE 1

### Valganciclovir 450 mg Tablets

| | |
|---|---|
| Ingredient | mg/Tablet |

Intragranular

| | |
|---|---|
| Valganciclovir hydrochloride | 496.3 |
| Microcrystalline cellulose | 53.7 |
| Crospovidone (Polyplasdone ® XL10) | 12 |

Granulation

| | |
|---|---|
| Povidone K-30 | 20 |
| Methanol* | q.s. |

Extragranular

| | |
|---|---|
| Crospovidone (Polyplasdone XL10) | 12 |
| Stearic acid | 6 |

Coating

| | |
|---|---|
| Opadry ® AMB Pink# | 24 |
| Methanol and dichloromethane* | q.s. |

*Evaporates during processing.
#Opadry ® AMB Pink is a formulated coating product from Colorcon that contains polyvinyl alcohol, titanium dioxide, macrogol/PEG 3350, talc, and iron oxide red.

Manufacturing Procedure:

1. Intragranular valganciclovir hydrochloride, microcrystalline cellulose, and crospovidone are passed through a #40 mesh sieve and mixed.

2. Povidone is dispersed in methanol and used to granulate the dry mixture.

3. The granules are dried to achieve a loss on drying (LOD) less than 2% w/w.

4. Dried granules are mixed with crospovidone, then with stearic acid.

5. The lubricated blend is compressed into tablets.

6. Compressed tablets are coated with a dispersion of Opadry® AMB Pink in a mixture of methanol and dichloromethane, to achieve a weight gain of 4% w/w after drying.

### EXAMPLE 2

### Valganciclovir 450 mg Tablets

| Ingredient | mg/Tablet |
|---|---|
| Valganciclovir hydrochloride | 496.3 |
| Microcrystalline cellulose (PH112) | 49.7 |
| Crospovidone (Polyplasdone XL10) | 12 |
| Povidone K-30 | 24 |
| Isopropyl alcohol* | q.s. |
| Crospovidone (Polyplasdone XL10) | 12 |
| Stearic acid | 6 |

Coating

| | |
|---|---|
| Opadry AMB Pink | 24 |
| Water* | q.s. |

*Evaporates during processing.

Manufacturing Procedure:

1. Valganciclovir hydrochloride, microcrystalline cellulose, crospovidone (first quantity), and povidone are passed through a #40 mesh sieve and mixed.

2. Isopropyl alcohol is sprayed onto the dry mixture to form granules.

3. The granules are dried to achieve a LOD less than 2% w/w.

4. Dried granules are mixed with crospovidone (second quantity), and then with stearic acid.

5. The lubricated blend is compressed into tablets.

6. Compressed tablets are coated with a dispersion of Opadry® AMB Pink in water to achieve a weight gain of 4% w/w after drying.

### EXAMPLE 3

### Valganciclovir 450 mg Tablets

| | |
|---|---|
| Ingredient | mg/Tablet |

Intragranular

| | |
|---|---|
| Valganciclovir hydrochloride | 496.3 |
| Microcrystalline cellulose | 53.7 |
| Crospovidone | 12 |

Granulation

| | |
|---|---|
| Povidone K-30 | 20 |
| Dichloromethane* | q.s. |

Extragranular

| | |
|---|---|
| Crospovidone | 12 |
| Stearic acid | 6 |

Coating

| | |
|---|---|
| Opadry AMB Pink | 24 |
| Dichloromethane* | q.s. |

*Evaporates during processing.

Manufacturing Procedure:

1. Intragranular valganciclovir hydrochloride, microcrystalline cellulose, and crospovidone are passed through a #40 mesh sieve and mixed.

2. Povidone is dispersed in dichloromethane and used to granulate the dry mixture.

3. The granules are dried to achieve a LOD less than 2% w/w.

4. Dried granules are mixed with extragranular crospovidone, and then with stearic acid.

5. The lubricated blend is compressed into tablets.

6. Compressed tablets are coated with a dispersion of Opadry® AMB Pink in dichloromethane to achieve a weight gain of 4% w/w after drying.

### EXAMPLE 4

### Valganciclovir 450 mg Tablets

| | |
|---|---|
| Ingredient | mg/Tablet |

Intragranular

| | |
|---|---|
| Valganciclovir hydrochloride | 496.3 |
| Microcrystalline cellulose | 53.7 |
| Crospovidone | 12 |

Granulation

| | |
|---|---|
| Povidone K-30 | 20 |
| Dichloromethane and methanol (1:1 by vol.)* | q.s. |

Extragranular

| | |
|---|---|
| Crospovidone (Polyplasdone XL10) | 12 |
| Stearic acid | 6 |

Coating

| | |
|---|---|
| Opadry AMB Pink | 24 |
| Methanol and dichloromethane* | q.s. |

*Evaporates during processing.

Manufacturing Procedure:

1. Intragranular valganciclovir hydrochloride, microcrystalline cellulose, and crospovidone are passed through a #40 mesh sieve and mixed.

2. Povidone is dispersed in a mixture of dichloromethane and methanol, and used to granulate the dry mixture.

3. The granules are dried to achieve a LOD less than 2% w/w.

4. Dried granules are mixed with extragranular crospovidone, and then with stearic acid.

5. The lubricated blend is compressed into tablets.

6. Compressed tablets are coated with a dispersion of Opadry® AMB Pink in a mixture of methanol and dichloromethane to achieve a weight gain of 4% w/w after drying.

### EXAMPLE 5

### Valganciclovir 450 mg Tablets

| | |
|---|---|
| Ingredient | mg/Tablet |

Intragranular

| | |
|---|---|
| Valganciclovir hydrochloride | 496.3 |
| Microcrystalline cellulose | 53.7 |
| Crospovidone | 12 |

Granulation

| | |
|---|---|
| Povidone K-30 | 20 |
| Ethyl acetate* | q.s. |

Extragranular

| | |
|---|---|
| Crospovidone (Polyplasdone XL 10) | 12 |
| Stearic acid | 6 |

Coating

| | |
|---|---|
| Opadry AMB Pink | 24 |
| Methanol and dichloromethane* | q.s. |

*Evaporates during processing.

Manufacturing Procedure:

1. Intragranular valganciclovir hydrochloride, microcrystalline cellulose, and crospovidone are passed through a #40 mesh sieve and mixed.

2. Povidone is mixed with ethyl acetate and used to granulate the dry mixture.

3. The granules are dried to achieve a LOD less than 2% w/w.

4. Dried granules are mixed with extragranular crospovidone, and then with stearic acid.

5. The lubricated blend is compressed into tablets.

6. Compressed tablets are coated with a dispersion of Opadry® AMB Pink in a mixture of methanol and dichloromethane to achieve a weight gain of 4% w/w after drying.

### EXAMPLE 6

### Valganciclovir 450 mg tablets

| | |
|---|---|
| Ingredient | mg/Tablet |

Intragranular

| | |
|---|---|
| Valganciclovir HCl (amorphous) | 496.3 |

Binder

| | |
|---|---|
| Povidone K-30 | 25 |
| Methylene chloride* | 250 |

Extragranular

| | |
|---|---|
| Crospovidone | 30 |
| Microcrystalline cellulose | 50.7 |
| Magnesium stearate | 3 |

Coating

| | |
|---|---|
| Opadry ® Pink YS-1-14519A | 18 |
| Methylene chloride* | 500 |

*Evaporates during processing.

Manufacturing Procedure:

1. Sift valganciclovir HCl through an ASTM #18 mesh sieve.

2. Dissolve povidone in methylene chloride.

3. Granulate the drug with povidone binder solution, then dry the granules.

4. Mill the dried granules to obtain the desired particle sizes.

5. Sift crospovidone and microcrystalline cellulose through an ASTM #18 mesh sieve.

6. Blend the materials of 5 with milled granules.

7. Sift magnesium stearate through an ASTM #40 mesh sieve and blend with the material of 6.

8. Compress the blend of 7 into tablets.

9. Coat the tablets with a dispersion of Opadry® Pink YS-1-14519A in methylene chloride, then dry.

### EXAMPLE 7

### Valganciclovir 450 mg Tablets

| | |
|---|---|
| Ingredient | mg/Tablet |

Intragranular

| | |
|---|---|
| Valganciclovir HCl (amorphous) | 496.3 |

Binder

| | |
|---|---|
| Povidone K-30 | 25 |
| Methylene chloride* | 250 |

Extragranular

| | |
|---|---|
| Crospovidone | 30 |
| Microcrystalline cellulose | 50.7 |
| Magnesium stearate | 3 |

Coating

| | |
|---|---|
| Opadry ® Pink YS-1-14519A | 18 |
| Methyl ethyl ketone* | 500 |

*Evaporates during processing.

Manufacturing Procedure:

1. Sift valganciclovir HCl through an ASTM #18 mesh sieve.

2. Dissolve povidone in methylene chloride.

3. Granulate the drug with povidone binder solution, then dry the granules.

4. Mill the dried granules to obtain desired particle sizes.

5. Sift crospovidone and microcrystalline cellulose through an ASTM #18 mesh sieve and blend with milled granules.

6. Sift magnesium stearate through an ASTM #40 mesh sieve and blend with the material of 5.

7. Compress the blend of 6 into tablets.

8. Coat the tablets with a dispersion of Opadry® Pink YS-1-14519A in methyl ethyl ketone, then dry.

### EXAMPLE 8

### Valganciclovir 450 mg Tablets

| | |
|---|---|
| Ingredient | mg/Tablet |

Intragranular

| | |
|---|---|
| Valganciclovir HCl (amorphous) | 496.3 |

Binder

| | |
|---|---|
| Povidone K-30 | 25 |
| Methylene chloride* | 250 |

Extragranular

| | |
|---|---|
| Crospovidone | 30 |
| Microcrystalline cellulose | 75.7 |
| Zinc stearate | 5 |

Coating

| | |
|---|---|
| Opadry ® Pink YS-1-14519A | 18 |
| Methylene chloride* | 500 |

*Evaporates during processing.

Manufacturing Procedure:

1. Sift valganciclovir HCl through an ASTM #18 mesh sieve.

2. Dissolve povidone in methylene chloride.

3. Granulate the drug with povidone binder solution, then dry the granules.

4. Mill the dried granules to obtain desired particle sizes.

5. Sift crospovidone and microcrystalline cellulose through an ASTM #18 mesh sieve and blend them with milled granules.

6. Sift zinc stearate through an ASTM #40 mesh sieve and blend with the material of 5.

7. Compress the blend of 6 into tablets.

8. Coat the tablets with a dispersion of Opadry® Pink YS-1-14519A in methylene chloride, then dry.

### EXAMPLE 9

### Valganciclovir 450 mg Tablets

| | |
|---|---|
| Ingredient | mg/Tablet |

Intragranular

| | |
|---|---|
| Valganciclovir HCl (amorphous) | 496.3 |

Binder

| | |
|---|---|
| Povidone K-30 | 25 |
| Methylene chloride* | 250 |

Extragranular

| | |
|---|---|
| Crospovidone | 30 |
| Microcrystalline cellulose | 50.7 |
| Magnesium stearate | 3 |

Seal Coating

| | |
|---|---|
| Hydrogenated vegetable oil | 4 |
| Talc | 3 |
| Methylene chloride* | 200 |

Film Coating

| | |
|---|---|
| Opadry ® Pink YS-1-14519A | 18 |
| Methylene chloride* | 500 |

*Evaporates during processing.

Manufacturing Procedure:

1. Sift valganciclovir HCl through an ASTM #18 mesh sieve.

2. Dissolve povidone in methylene chloride.

3. Granulate the drug with povidone binder solution, then dry the granules.

4. Mill the dried granules to obtain desired particle sizes.

5. Sift crospovidone and microcrystalline cellulose through an ASTM #18 mesh sieve and blend with milled granules.

6. Sift magnesium stearate through an ASTM #40 mesh sieve and blend with the material of 5.

7. Compress the blend of 6 into tablets.

8. Coat the tablets with a dispersion of Opadry® Pink YS-1-14519A in methylene chloride, then dry.

### EXAMPLE 10

### Valganciclovir 450 mg Tablets

| | |
|---|---|
| Ingredient | mg/Tablet |

Intragranular

| | |
|---|---|
| Valganciclovir HCl (amorphous) | 496.3 |

Binder

| | |
|---|---|
| Povidone K-30 | 25 |
| Methylene chloride* | 250 |

Extragranular

| | |
|---|---|
| Crospovidone | 30 |
| Microcrystalline cellulose | 50.7 |
| Magnesium stearate | 3 |

Seal Coating

| | |
|---|---|
| Sodium stearyl fumarate | 4 |
| Talc | 3 |
| Methanol* | 20 |
| Methylene chloride* | 180 |

Film Coating

| | |
|---|---|
| Opadry ® Pink YS-1-14519A | 18 |
| Methylene chloride* | 500 |

*Evaporates during processing.

Manufacturing Procedure:

1. Sift valganciclovir HCl through an ASTM #18 mesh sieve.

2. Dissolve povidone in methylene chloride.

3. Granulate the drug with povidone binder solution, then dry the granules.

4. Mill the dried granules to obtain desired particle sizes.

5. Sift crospovidone and microcrystalline cellulose through an ASTM #18 mesh sieve and blend with milled granules.

6. Sift magnesium stearate through an ASTM #40 mesh sieve and blend with the material of 5.

7. Compress the blend of 6 into tablets.

8. Coat the tablets with a dispersion of Opadry® Pink YS-1-14519A in methylene chloride, then dry.

1. A solid dosage form comprising valganciclovir and one or more pharmaceutical acceptable excipients, being prepared by a process comprising non-aqueous solvent or hydro-alcohol granulation.

2. The solid dosage form of claim 1, wherein granulation comprises mixing valganciclovir or a salt thereof, at least one pharmaceutical acceptable excipient, and a non-aqueous solvent or hydro-alcohol.

3. The solid dosage form of claim 1, wherein a non-aqueous solvent is methanol, ethanol, isopropyl alcohol, dichloromethane, ethyl acetate, or a mixture of two or more thereof.

4. The solid dosage form of claim 1, wherein granulation is performed using a solution of a binder in a non-aqueous solvent.

5. The solid dosage form of claim 4, wherein a binder is one or more of a polyvinylpyrrolidone, hydroxypropyl cellulose, hydroxypropyl methylcellulose, starch, gelatin, or gum.

6. The solid dosage form of claim 1, wherein granulation is performed using a solution of a binder in a hydro-alcohol.

7. The solid dosage form of claim 6, wherein a binder is one or more of a polyvinylpyrrolidone, hydroxypropyl cellulose, hydroxypropyl methylcellulose, starch, gelatin, or gum.

8. The solid dosage form of claim 1, wherein valganciclovir is provided by amorphous valganciclovir hydrochloride.

9. The solid dosage form of claim 1, which is a tablet.

10. The solid dosage form of claim 9, wherein a tablet is provided with a polymer film coating, and optionally further coated with a hydrophobic coating.

11. The solid dosage form of claim 1, comprising a particulate material contained in a capsule.

12. A solid dosage form comprising amorphous valganciclovir hydrochloride and one or more pharmaceutical acceptable excipients, being prepared by a process comprising granulation with a mixture of a non-aqueous solvent and a binder.

13. The solid dosage form of claim 12, wherein a binder comprises one or more of a polyvinylpyrrolidone, hydroxypropyl cellulose, hydroxypropyl methylcellulose, starch, gelatin, or gum.

14. The solid dosage form of claim 12, wherein a non-aqueous solvent is methanol, ethanol, isopropyl alcohol, dichloromethane, ethyl acetate, or a mixture of two or more thereof.

15. The solid dosage form of claim 12, which is a tablet.

16. The solid dosage form of claim 15, wherein a tablet is provided with a polymer film coating, and optionally further coated with a hydrophobic coating.

17. A process for preparing a solid dosage form, comprising mixing valganciclovir hydrochloride with at least one pharmaceutically acceptable excipient and granulating by spraying or adding a non-aqueous solvent or hydro-alcohol.

18. The process of claim 12, wherein a non-aqueous solvent or hydro-alcohol is combined with a binder.

19. The process of claim 12, wherein valganciclovir hydrochloride is in an amorphous form.
